(19) United States
(12) Patent Application Publication
(10) Pub. No.: US 2004/0153145 A1
(43) Pub. Date: Aug. 5, 2004

(54) Fixation method for bioprostheses

(75) Inventors: Dan T. Simionescu, Central, SC (US); Narendra Vyavahare, Easley, SC (US)

Correspondence Address: DORITY & MANNING, P.A., POST OFFICE BOX 1449, GREENVILLE, SC, 29602-1449, US

(73) Assignee: Clemson University

(21) Appl. No.: 10/722,142

(22) Filed: Nov. 24, 2003

Related U.S. Application Data
(60) Provisional application No. 60/429,190, filed on Nov. 26, 2002.

(51) Int. Cl. A61F 2/24

(52) U.S. Cl. 623/2.14; 623/918; 8/94.11

(57) ABSTRACT

An improved fixative for tissue useful for bioprosthetic heart valves is provided. The tissue can have an elastin content and the elastin can be chemically fixed using a phenolic tannin, for example, tannic acid. The fixed elastin component provides greater mechanical durability and improved resistance to biological degradation following implantation. The tannic acid fixation protocol allows for biological material having a high elastin content, for example, about 30% or more. When used in combination with a glutaraldehyde fixative an additive effect can be seen in increased cross-link density and increased resistance to degradation and calcification.

### CROSS REFERENCE TO RELATED APPLICATION

This application claims benefit to U.S. Provisional Application serial No. 60/429,190 filed Nov. 26, 2002.

### FEDERALLY SPONSORED RESEARCH AND DEVELOPMENT

The United States Government may have rights in this invention pursuant to Grant No. HL 61652 between Clemson University and the National Institutes of Health.

### BACKGROUND OF THE INVENTION

Prosthetic heart valves are used to replace damaged or diseased heart valves. Prosthetic heart valves may be used to replace a heart's natural valves including aortic, mitral, and pulmonary valves. The predominant types of prosthetic heart valves are either mechanical valves or bioprosthetlic valves. Bioprosthetic valves include allograft valves, which include tissue supplied from human cadavers; autologous valves, which include tissue of the individual receiving the valve; and xenograft valves, which include tissue obtained from non-human biological sources such as pigs, cows, or other animals.

Presently, mechanical valves have the longest durability of available replacement heart valves. However, implantation of a mechanical valve requires a recipient to be prescribed anticoagulants to prevent formation of blood clots. Unfortunately, continuous use of anticoagulants can be dangerous, as it greatly increases the user's risk of serious hemorrhage. In addition, a mechanical valve can often be audible to the recipient and may fail without warning, which can result in serious consequences, even death.

The use of bioprosthetic heart valves (BHVs) in valve replacement procedures is often preferred as BHVs do not require ongoing patient treatment with anticoagulants. Allograft transplants have been quite effective, with good compatibility and blood flow characteristics in the recipients. However, the availability of human valves for transplantation continues to decline as a percentage of cardiac surgeries performed each year. As such, the choice of xenograft materials for use in replacement BHVs is becoming more common.

Both xenografts and allografts require that the graft tissue be chemically fixed, or cross-linked, prior to use, in order to render the tissue non-antigenic as well as improve resistance to degradation. Currently, glutaraldehyde fixation of xenograft and allograft tissue is commonly used. Glutaraldehyde fixation forms covalent cross-links between the free amines of certain tissue proteins. As a result, the tissue is less susceptible to adverse immune reactions by the patient. Fixation is also believed to improve the valve durability.

One disadvantage of current xenograft materials however, remains durability. At present, conventional xenograft valves require replacement within five to ten years of the original repair. This is at least in part due to the fact that xenografts are stiffer and less pliable than the recipient's original healthy tissue. As a consequence of the increased stiffness, the periodic opening and closing of the valve leads to material fatigue of the bioprosthetic replacement tissue. In addition, the recipient's heart will be required to work harder to overcome the stiffness of a bioprosthetic valve as compared to the exertion required for the original valve to function. As the material integrity of the xenograft valve is lessened over time, the efficiency of the valve operation also decreases. Additionally, fatigue and mechanical degradation of the xenograft valve is associated with increased calcification of the valve. The calcification causes additional stiffening which further degrades the physical and biological integrity of the valve.

The degeneration of fixed biological tissues used in BHVs is considered one of the major causes of long-term failure of such implants. Despite advances in producing longer lasting and better performing heart valves, there remains room for variation and improvement within the art.

### SUMMARY OF THE INVENTION

The present invention is directed to a method for fixing a tissue for use in a bioprosthetic and the bioprostheses that include the fixed tissue. In one embodiment, the method includes providing a tissue comprising elastin and fixing the tissue with a solution comprising a phenolic tannin. The fixed tissue can then have an elastin component that is substantially resistant to biodegradation.

The tissue can also include a collagen component. In one embodiment, the process can include fixing the tissue with a glutaraldehyde solution, which can enhance the stabilization of the collagen component in the tissue. For example, in one embodiment, the tissue can first be fixed with the glutaraldehyde fixative and subsequently be fixed with the phenolic tannin fixative.

The tissue can be any suitable bioprosthetic tissue. For example, the tissue can be a xenograft material. For instance, the tissue source can be a bovine source or a porcine source. In one embodiment, the tissue can be pericardial, aortic wall (e.g. aortic arch), heart valve, or vena cava tissue.

The phenolic tannin used to fix the elastin component of the tissue can be, in one embodiment, a tannic acid. For instance, the solution can include tannic acid in a concentration between about 0.0001 grams per 100 milliliters of solution (g/100 ml) to about 10 g/100 ml. In one embodiment, the solution can include tannic acid in a concentration between about 0.3 g/100 ml and about 1.0 g/100 ml. In addition, the solution can include a buffer. The solution can, in one embodiment, be at a pH of less than about 6.

Due to the ability to stabilize the elastin component of the tissue utilizing the disclosed fixatives, the tissue can optionally have a relatively high elastin content. For instance, the tissue can have at least about 10% elastin content by weight in certain embodiments.

In addition to the elastin component of tissue, the disclosed fixatives can also fix other tissue components not fixed by glutaraldehyde fixatives used in the past. For example, the disclosed phenolic tannins can also fix glycosaminoglycan polysaccharides in the tissue.

The fixed tissue of the present invention can be incorporated into a bioprosthesis according to methods as are generally known in the art, and thus are not discussed in detail herein. For example, the fixed tissue can be attached to a variety of support materials according to methods generally known in the art and utilized for other tissues in the past. Support materials can include, for example, stents or suture rings. In one embodiment, the fixed tissue can be utilized in a bioprosthetic heart valve.

Optionally, the fixed tissue can be an anisotropic tissue. Due to the improved stabilization of the tissue components afforded by the disclosed fixatives and fixation protocols herein, the anisotropic characteristics of the fixed tissue can be maintained following formation and implantation of bioprostheses. As such, in one embodiment, the fixed tissue can be oriented within the bioprosthetic so as to more closely mimic the characteristics of the tissue which is being replaced by the disclosed fixed tissues. For instance, the fixed tissue can have increased elasticity in a direction, and the tissue can be oriented with that direction of increased elasticity within the bioprosthesis so as to more closely mimic the elastic characteristics of the replaced tissue.

The fixed tissue of the disclosed invention, including elastin cross-linked by a phenolic tannin cross-linking agent, can exhibit improved degradation characteristics as compared to fixed tissues utilized in bioprosthetics in the past. For example, the fixed tissue can have a temperature of thermal denaturation of greater than about 70° C. In one embodiment, the fixed tissue can have a temperature of thermal denaturation greater than about 80° C.

The fixed tissue of the present invention can also exhibit good durability in the presence of proteins which can degrade elastin, such as elastase. For example, the fixed tissue of the present invention can exhibit less than about 20% degradation following exposure to elastase for a period of about 48 hours.

The fixed tissue of the present invention can also exhibit less calcification over time as compared to tissue fixed with glutaraldehyde fixatives known in the past. For example, the fixed tissue of the present invention can exhibit at least about 60% less calcification over time as compared to a similar tissue fixed with only a glutaraldehyde fixative.

The present invention is also directed to methods of replacing damaged heart valves with bioprosthetic heart valves including the tissue as herein disclosed.

### BRIEF DESCRIPTION OF THE FIGURES

A full and enabling disclosure of the present invention, including the best mode thereof, to one of ordinary skill in the art, is set forth more particularly in the remainder of the specification, including reference to the accompanying drawings in which:

FIG. 1 is a graph indicating the thermal denaturation temperature of various xenograft materials before and after fixation with glutaraldehyde;

FIG. 2 is a graph indicating the thermal denaturation temperature of pericardium tissue setting forth comparative results of various fixation protocols;

FIG. 3 is a graph indicating relative percent degradation of aortic wall using elastase and comparing various types of fixatives;

FIG. 4 is a comparative graph showing relative amounts of collagen and elastin in various source tissues; and

FIG. 5 is a comparative graph showing the degradation effect of elastase on elastin fixed with different fixatives.

### DETAILED DESCRIPTION OF THE INVENTION

Reference now will be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. Thus, it is intended that the present invention cover such modifications and variations as come within the scope of the appended claims and their equivalents. Other objects, features, and aspects of the present invention are disclosed in the following detailed description. It is to be understood by one of ordinary skill in the art that the present discussion is a description of exemplary embodiments only and is not intended as limiting the broader aspects of the present invention, which broader aspects are embodied in the exemplary constructions.

As used herein the term “bioprosthesis” includes any prosthesis which is derived in whole or in part from human, animal, or other organic tissue and which can be implanted into a human or an animal. Accordingly, the term “bioprosthesis” includes cardiac prostheses such as heart valves, other replacement heart components, and cardiac vascular grafts. In addition, the properties of the tissue described herein may also lend itself as a prosthetic material for use with other organs and tissue systems.

As used herein, the term “cross-link” refers generally to the process of forming bonds, e.g., covalent bonds, between free, active moieties on or within tissue or between a cross-linking agent or other compound which reacts with a reactive moiety of the tissue. It is generally recognized that in forming bioprostheses, it is desirable to leave as few active moieties within the biological tissue as possible. The resulting cross-linked tissue is considered “fixed.”

As used herein, the term “fixed” in regard to tissue is defined to refer to tissue that is stabilized so as to be less antigenic and less susceptible to physical and biological degradation.

The term “tissue” is used as understood by those having skill in the art to include any natural or synthetic material derived from an organic source and which may be implanted in a mammal. While exemplary forms of a tissue are described herein, the term “tissue” is not limited to the exemplary embodiments but may include other types of tissues having properties similar to the exemplary tissue.

In general, the present invention is directed to an improved tissue fixative, fixation protocol, and a resulting fixed tissue for use in bioprostheses, including, for instance, bioprosthetic heart valves. More specifically, the fixative of the present invention can improve stabilization of the elastin component within tissue as compared to tissue fixatives known in the past. In one preferred embodiment, the fixative of the present invention includes a tannic acid (TA). The disclosed fixatives have been found to increase the stability of elastin within tissues with respect to tissue-degrading enzymes. This increased stability can reduce the propensity of the tissues, which can be used to form any of a variety of bioprostheses, to undergo biological and mechanical degradation. The fixed biological material prepared according to the disclosed processes may be used to form bioprostheses that, as a result of the improved materials, can exhibit improved properties of strength, durability, and elasticity.

The fixed tissues of the present invention can generally be utilized in any of a number of bioprostheses. For instance, tissue fixed according to the present invention can be utilized in forming any of a variety of cardiac bioprostheses that can replace damaged sections of the cardiovascular system. For example, bioprosthetic heart valves, veins, or arteries can be formed. In general, the bioprostheses of the present invention can include the fixed tissue materials herein discussed in conjunction with other support materials as are generally known in the art. For instance, bioprostheses according to the present invention can include the disclosed fixed tissue in suitable combination with support materials such as wire forms, stents, suture rings, conduits, flanges, and the like.

In one embodiment, a BHV can be formed including heart valve leaflets formed of the disclosed tissues and secured to a stent. Suitable stent materials can generally include stent materials as may generally be found in other known heart valves, including both mechanical and bioprosthetic heart valves. For example, in one embodiment, tissue leaflets that have been fixed according to the present invention can be attached to a flexible polymer stent formed of, for example, polypropylene, and reinforced with a metal ring (such as, for example, a Haynes™ alloy no. 25 metal ring). In another embodiment of the invention, a polymer stent can be used including a polyester film support secured to a surgically acceptable metal ring such as an Elgiloy™ metal stiffener. Optionally, a stent may be formed of only polymeric materials, and not include any metals. Alternatively, the disclosed bioprosthesis can include a wire stent, such as an Elgiloy™ wire stent, or a titanium stent, which can be optionally covered with a material cover, such as, for example, Dacron™. In some embodiments, the bioprosthesis can also include a sewing or suture ring such as, for example, a polyester, Dacron™, or Teflon™ suture ring, as are generally known in the art. In yet another embodiment, the disclosed bioprosthesis can be a stentless heart valve. It should be clear, however, that these are exemplary materials, and the make-up of the support material used in combination with the disclosed fixed tissues is not critical to the disclosed invention.

Following formation of a bioprosthetic device according to the present invention, the device can be implanted by any surgical procedure as is generally known in the art. For example, a BHV including the tissue of the invention can be implanted in the heart of a person or an animal according to known surgical procedures such as, for example, procedures described in U.S. Pat. No. 6,532,388 to Hill, et al., U.S. Pat. No. 6,506,197 to Rollero, et al., and U.S. Pat. Nos. 6,402,780, 6,042,607, and 5,716,370 all to Williamson. IV, et al., all of which are incorporated herein by reference. In general, such procedures include removal of a damaged cardiac valve, implantation of the new replacement valve in the cardiac valve annulus, and attachment of the BHV to the adjacent tissue.

The improved fixative of the present invention can be utilized to fix any suitable bioprosthetic tissue including xenograft or allograft materials. In general, suitable tissues can be provided by tissue culture techniques as are generally known in the art, and thus, such techniques need not be discussed in detail herein.

Connective tissues such as may be utilized as source materials for the bioprostheses of the present invention in general contain both collagen and elastin. Collagen and elastin are protein constituents of connective tissue that together are primarily responsible for the strength, elasticity and integrity of the tissue. Collagen is the fibrous protein constituent of connective tissue. Chemically, it is a triple helix formed of three extended protein chains that wrap around one another. In vivo, many rod-like collagen molecules are cross-linked together in the extracellular space to form unextendable collagen fibrils that have the tensile strength of steel. Elastin is a protein that is somewhat similar to collagen in make-up and is the principal structural component of elastic fibers. Elastin polypeptide chains are cross-linked together to form rubber-like, elastic fibers. Unlike collagen, elastin molecules can uncoil into a more extended conformation when the fiber is stretched and will recoil spontaneously as soon as the stretching force is relaxed.

In the past, glutaraldehyde has been the common fixative used to stabilize and fix tissue for bioprosthetic applications. Glutaraldehyde fixation forms covalent cross-links between free amines in certain tissue proteins, primarily collagen. Elastin, in contrast, lacks the free amine groups that provide the principal form of interaction with glutaraldehyde. As such, while glutaraldehyde can provide suitable fixation of the collagen in a connective tissue, the elastin is not likewise fixed. As a result, connective tissues containing a relatively large percentage of collagen have often been chosen to form bioprostheses in order to improve the overall cross-link density of the fixed tissue. Unfortunately, tissues containing a relatively greater amount of collagen can be much stiffer and less pliable than tissues containing a relatively greater elastin content, and the resulting fixed tissues can be equally stiff and un-pliable, leading to the problems of the bioprostheses of the past, discussed above. In addition, as the elastin content of the tissue is not stabilized by the standard glutaraldehyde processes, the elastin that is in the tissue can be more susceptible to biological degradation over time, and the tissue can lose what pliability and elasticity it does have over the life of the prosthesis.

The fixative and fixation protocol disclosed by the present invention improves stabilization of additional protein components of the tissues not stabilized by glutaraldehyde fixatives, and in particular improves stabilization of the elastin component. In accordance with this invention, it has been found that use of fixatives that can cross-link components of the tissue that are not stabilized by glutaraldehyde can not only improve the strength and durability of tissues utilized in bioprostheses in the past, but can also provide a process for utilizing tissues not previously considered feasible for bioprostheses. For instance, the disclosed processes can be utilized to stabilize high elastin-content tissue that can then be utilized to form durable, pliable bioprostheses.

In general, the fixatives of the present invention can include phenolic tannin fixatives. In one preferred embodiment, the fixative can include a tannic acid component. Other fixative agents are also encompassed according to the present invention, however. For example, other tannin compounds including gallotannis, catechins, flavonoids, and derivatives thereof can be utilized in the fixative compositions of the present invention.

Tannic acid is a naturally derived polyphenol that can cross-link proteins by the formation of multiple hydrogen bonds. Properties of tannic acid may be found in reference to the publication Plant Polyphenols, Cambridge University Press, Cambridge U.K., 1989, pp. 123-195, which is incorporated herein by reference.

Tannic acid, as a cross-linking agent, is similar in many properties to that of previously known fixatives, including glutaraldehyde fixatives. For example, tannic acid is known to cross-link with collagen. In addition, tannic acid has been used as an elastin stain for electron microscopy, and has been used as a contrast-increasing agent for collagen staining. Additionally, tannic acid is known to have antibacterial properties, can inhibit enzymes, and can reduce protein antigenicity.

Unlike glutaraldehyde, however, tannic acid can interact with elastin as well as other connective tissue components. For instance, tannic acid is capable of cross-linking glycosaminoglycan polysaccharides and other connective tissue components not amenable for glutaraldehyde fixation. Specifically, tannic acid is believed able to interact with elastin through proline-rich areas within the elastin matrix molecules. In accordance with the present invention, it has been found that tannic acid is useful as an elastin fixative in formation of bioprosthetic materials such as may be used for bioprosthetic heart valves. Accordingly, in one embodiment, the present invention allows an additional level of stabilization of bioprosthesis tissue components by combining the fixation abilities of glutaraldehyde with the additional ability of tannic acid.

In one embodiment, buffered tannic acid solutions having a pH of less than about 6 can be used as a fixative agent in which the tannic acid concentration can vary from about 0.3 g/100 ml to about 1.0 g/100 ml. It should be noted, however, that while these exemplary concentrations are effective, it is believed that a wide range of tannic acid concentrations may be employed in the fixatives of the present invention. For example, actual concentrations used may be influenced by the type of tissue, thickness of tissue, desired incubation time, and preferred pH. As such, in certain embodiments of the present invention, concentrations of tannic acid ranging from about 0.0001 g/000 ml to about 10 g/100 ml may be useful.

Similarly, while buffered pH solutions of 7.4 have been used in the fixation protocols described below, it is believed that a wider range of pHs may optionally be used. For example, a solution having a pH from about 4.0 to about 9.0 may be used in conjunction with a variety of different buffers including phosphate buffers, borate buffers, HEPES, PIPES, and MOPSO. In one embodiment, a solution having a pH of less than about 6 can be preferred. It is also believed that a wide variation in fixation time ranging from, for example, about 24 hours to 7 days or even greater may be operative. Likewise, fixation temperatures may also vary. In one embodiment, fixation temperatures may vary between about 20° C. and about 40° C., although greater and lesser temperatures are also envisioned in that there is no known criticality to temperature regimes typically used for fixing biological materials, provided, of course, that the biological materials are not destroyed by the process.

The fixatives of the present invention, which can cross-link protein components not cross-linked by protocols utilized in the past, can provide a fixed tissue in which the total cross-link density of the tissue may be increased as compared to fixed tissues prepared in the past. Specifically, the cross-linking agents of the disclosed fixatives can target and cross-link molecules which are largely unaffected by conventional glutaraldehyde-based fixation protocols, including elastin. In addition, the fixatives of the present invention can cross-link these molecules with no detrimental effect on the ability of the fixative to cross-link the other components in the tissue, i.e., the collagen component. In fact, it is believed that the disclosed fixative agents can, in certain embodiments, not only exhibit no detrimental effect on the ability to cross-link these components, but can also have an additive effect when used in conjunction with other known agents and can increase the cross-linking density of collagen components as well as the elastin and elastin-type components.

For example, in one embodiment, the disclosed fixative compositions can be utilized to fix a collagen-rich natural tissue, for instance a pericardial tissue. In this embodiment, the tissue may first be fixed with a known glutaraldehyde fixative, which can cross-link the collagen components of the tissue. Following the glutaraldehyde process, the tissue can be treated with the disclosed fixatives. As described in more detail in the example section below, in this embodiment, it is believed that the disclosed agents can cross-link not only tissue components not fixed by the glutaraldehyde fixative, for example the elastin components, but can also cross-link additional collagen components not already cross-linked by the glutaraldehyde. Though not wishing to be bound by any theory, it is believed that the later fixation process and composition can cross-link additional sites in the tissue to which the glutaraldehyde fixative has no access.

Similarly, when fixing tissue having a higher elastin content, such as porcine aortic wall tissue, the combination of a glutaraldehyde fixative agent with a phenolic tannin agent such as tannic acid can have an additive effect with respect to increased cross-link density as compared to when either cross-linking agent is utilized alone.

As a result of the disclosed fixation protocol, the resulting source material can exhibit improved cross-link density when compared to a fixation protocol utilizing only glutaraldehyde as the cross-linking agent. In one embodiment, the present invention allows conventional bioprosthetic heart valve materials, such as pericardium and aortic cusp or aortic arch materials, to achieve even greater cross-link density by preserving the elastin component that was not fixed by previous methods as well as, in certain embodiments as described above, improving the cross-link density of the collagen component. As such, the mechanical properties of the elastin component of these materials can be better maintained over the life of the bioprosthesis. Further, physical fatigue and calcification associated with in vivo use of pericardium and aortic tissue has been shown to be lessened by use of the disclosed fixatives, and as described further in Example 5, below.

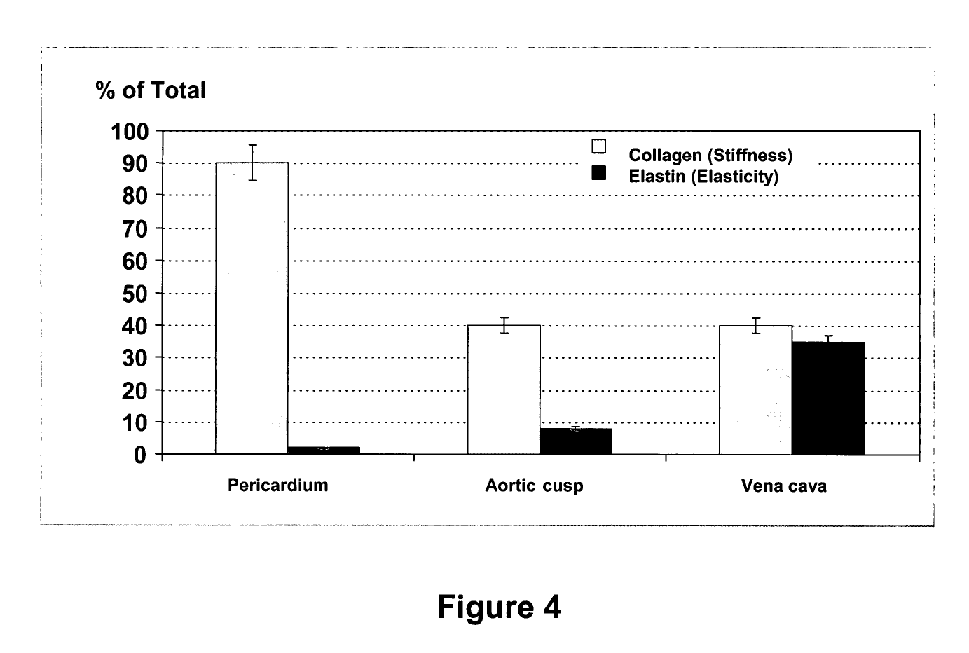

As seen in reference to FIG. 4, the elastin content of various source tissue such as may be found in, for example, BHVs, is provided. Exemplary source materials shown include pericardium, aortic cusp, and vena cava material. In forming xenograft materials, tissue such as those illustrated can generally be provided from porcine, bovine or similar large animals. Tissues may, however, optionally be provided from allograft materials, as is known in the art. As illustrated in FIG. 4, source tissues can have significant variations in the relative amounts of collagen and elastin found in the material. Due to the nature of both elastin and collagen, the relative amount of elastin material as compared to collagen can impact not only the physical properties of the tissue in vivo, but can also affect the long term in vivo durability of the tissue following fixation and utilization as a bioprosthetic material. For example, as can be seen in reference to FIG. 4, pericardial tissue contains about 90% by weight collagen and only about 2% by weight elastin. Thus, pericardial tissue, while very strong and resilient, is not particularly pliable or elastic. Similarly, the fixed pericardial tissue will not exhibit a great deal of elasticity and, when utilizing a fixative which does not substantially stabilize the elastin content of the tissue, stiffness of the bioprosthesis can increase as what elastin there is will degrade over time, which can lead to the problems discussed above.

The fixed tissues of the present invention can exhibit increased elasticity while rendering the elastin component less susceptible to biodegradation as well as to the resulting degradation and calcification of the bioprosthesis. In addition, the ability to improve the chemical fixation of tissue components, and primarily elastin, permits the use of high elastin content tissues as a tissue source. Such tissues were, heretofore, considered undesirable in that the high elastin content diminished the long-term integrity of the bioprosthesis due to the inability to fix the elastin component of the tissue. The disclosed fixation method, however, increases the stability of elastin and elastin-rich tissues against degrading enzymes. As a result, elastin-rich tissues, heretofore undesirable because of the inability of glutaraldehyde to stabilize the elastin components, may now be used as a source of tissue. As such, the inherent properties associated with high elastin content tissues, such as increased elasticity and anisotropic properties, may be used to advantage in selecting and orienting a tissue suitable for use in bioprostheses and, specifically, in replacement BHVs. The resulting fixed tissue can offer improvements over conventional xenografts or allografts of pericardium-derived or other source tissue.

For example, in one embodiment, the fixative of the present invention can be utilized to fix tissues containing relatively high levels of elastin. In one embodiment, a fixed tissue suitable for bioprosthetic replacement of cardiac tissue can be prepared, the source tissue having an elastin content greater than about 10% by weight. In one embodiment, a fixed tissue suitable for bioprosthetic replacement of cardiac tissue can be prepared, the tissue having an elastin content of at least about 30% by weight.

In one particular embodiment of the present invention, high elastin content materials such as vena cava tissue can be fixed according to the disclosed processes and utilized as a source tissue for bioprosthetics including BHVs. The useful nature of vena cava derived source tissue is reflective of the molecular and structural composition of the tissue. As seen in reference to FIG. 4, a comparison of the tissue composition of elastin and collagen is provided for pericardium, aortic cusps, and vena cava. As seen, the vena cava material has only about 40% by weight of collagen compared to a 90% value for pericardium. Additionally, the vena cava material has a much higher percentage of elastin. The combination of increased elastin content and decreased collagen content can contribute to the improved properties of the resulting tissue, e.g., lasting pliability and elasticity leading to reduced calcification over time.

In one embodiment, the fixative of the present invention can include both glutaraldehyde and tannic acid components in combination in a fixation protocol for a high elastin containing tissue, such as vena cava tissue. According to this embodiment, the resulting fixed tissue can have three to four times greater elasticity than similarly fixed tissue derived from the pericardium of the same donor species. The greater extensibility of the vena cava material is believed to offer long-term benefits in terms of durability and resistance to mechanical degradation. The increase in mechanical durability can also provide additional attributes in terms of reducing the onset and amount of calcification which is frequently associated with bioprosthetic heart valve failure. Additionally, to the extent that the more elastic tissue is resistant to mechanical damage and degradation, it is believed that greater resistance to biological degradation is also provided. Both the resistance to calcification and resistance to biological degradation are each believed to further enhance the longevity of implanted bioprostheses of the present tissue.

In one embodiment, the tissue of the present invention can be an anisotropic material and can more closely mimic the natural action and elasticity of the replaced organ or tissue. For example, an anisotropic fixed biological material can be prepared that has an elastin component which provides greater stiffness in one direction and a greater elasticity in a cross direction.

In one embodiment of the present invention, pericardial tissue can be fixed and used to construct bioprostheses such as pulmonary valves, aortic valves, mitral valves, or aortas. Pericardium material is an anisotropic material, and can have variations in physical properties. For example, Simionescu et al. (Mapping of Glutaraldehyde-treated Bovine Pericardium and Tissue Selection for Bio-Prosthetic Heart Valves, Journal of Biomedical Materials Research, 27(6):697, 1993, which is incorporated herein by reference) discusses differences in individual pericardium sacs with respect to fiber orientation, suture holding power, and thickness. According to the present invention, these anisotropic qualities can be preserved through the disclosed fixation process, as the different proteins that provide the anisotropic characteristics to the native tissue can be preserved, thereby preserving the associated characteristics. In the past, when only certain elements of the tissue were preserved through the cross-linking process, some of the associated anisotropic characteristics could also be lost. In the present invention, however, the anisotropic qualities can be preserved. The resulting fixed anisotropic tissue can then be oriented when forming the bioprosthesis so as to more closely mimic the anisotropic characteristics of the natural material that is being replacing.

As discussed above, in one embodiment, vena cava derived tissue may be used in the construction of valves and bioprosthetic heart components. Vena cava tissue, similar to pericardial tissue, is an anisotropic material. Somewhat different than pericardial tissue, however, the anisotropic characteristics of vena cava tissue can be more regular with regard to the orientation of the tissue. Specifically, the anisotropic properties of the vena cava derived tissue can include greater elasticity in one direction and greater stiffness in another direction. Thus, the fixed tissue can be positioned and oriented within a bioprosthetic so as to achieve enhanced mechanical performance. For example, the anisotropic tissue can be oriented in the bioprostheses so as to exhibit a greater stiffness in one direction, preferably the direction that will require less movement following implant, and to exhibit greater elasticity in the direction in which the tissue will generally be expected to move following implant. As such, even greater improvements in mechanical characteristics can be obtained in the present invention in bioprostheses prepared with anisotropic fixed tissues. The enhanced mechanical performance is believed to afford greater longevity of the bioprostheses, thereby reducing the occurrence of subsequent surgery to repair a damaged or failing prosthesis.

Reference now will be made to exemplary embodiments of the invention set forth below. Each example is provided by way of explanation of the invention, not as a limitation of the invention. In fact, it will be apparent to those skilled in the art that various modifications and variations may be made of this invention without departing from the scope or spirit of the invention.

While the examples below are described in reference to porcine inferior vena cava material, it is believed that porcine superior vena cava material will also provide the benefits as noted below. Additionally, to the extent tissue derived from other animal species provides similar benefits, the scope of the present disclosure and claims should not be limited to tissue derived from any one species. Further, to the extent tissue can be provided by either tissue culture or grafts, such tissues are believed useful as a tissue as set forth in this present invention.

### EXAMPLE 1

Pure elastin labeled with orcein was used as a substrate for the enzyme elastase. The labeled elastin substrate was used per se, as well as labeled elastin fixed separately with glutaraldehyde (GA) and with tannic acid (TA).

Purified insoluble elastin labeled with orcein was treated for 24 hours at room temperature with one of:

0.6% GA in 50 mM Hepes buffered saline at pH 7.4 (GA-fixed elastin);

0.3% TA in 50 mM Hepes buffered saline at pH 7.4 (TA-fixed elastin); or

50 mM Hepes buffered saline at pH 7.4 (Buffer control).

Elastin samples were centrifuged at 3000 rpm for 10 minutes at room temperature, rinsed with double distilled (dd)H₂O and dialyzed in ddH₂O. Treated elastin was suspended in elastase buffer (50 mM Tris, 1 mM CaCl₂, 0.02% NaN₃, pH 7.8) at a concentration of 20 mg/ml. A blank of labeled elastin incubated in the absence of elastase was also prepared for comparison.

Pure pancreatic elastase was prepared at a concentration of 1 Unit/ml in elastase buffer (described above), mixed with treated elastin samples in a 1 to 1 ratio and incubated at 37° C. for 3 days. Samples were centrifuged and elastin degradation was assessed by measuring the presence of soluble orcein-labeled elastin peptides in supernatants, by measuring optical density at 570 nm.

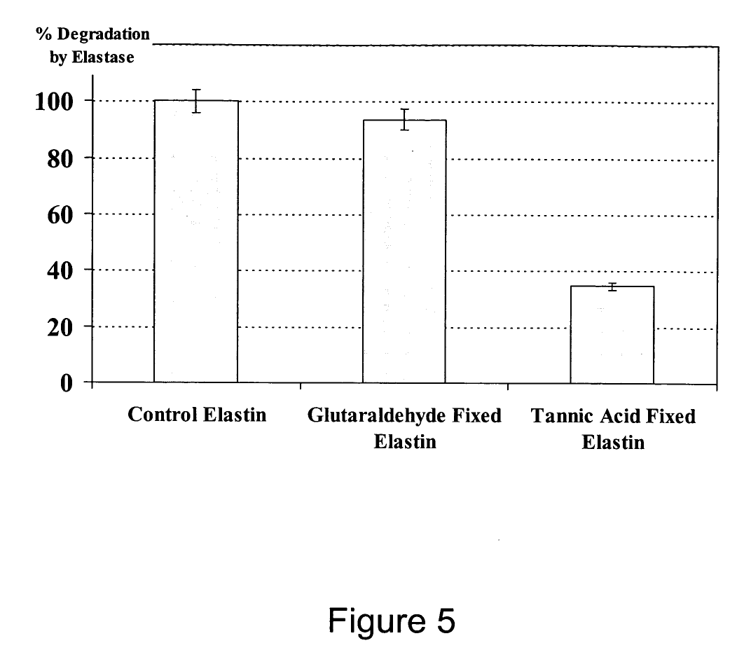

Under these experimental conditions, untreated (buffer control) elastin was completely degraded by elastase. As described in reference to FIG. 5 and to Table 1 below, GA has a minor effect on elastin degradation (reduction of 6.7%) while TA-treated elastin was rendered significantly resistant to degradation by elastase (65% reduction in susceptibility to degradation).

TABLE 1
Effect of TA and GA on degradation of pure elastin

| Treatment | % Elastin degradation | SEM (n = 6) |
|---|---|---|
| Buffer control | 100 | 0.012 |
| GA fixed elastin | 93.7 | 0.004 |
| TA fixed elastin | 34.6 | 0.001 |

The results indicate that GA does not protect elastin adequately from enzymatic degradation and that TA is more effective in reducing the susceptibility of elastin towards degradation by elastase.

### EXAMPLE 2

Porcine aorta fragments were collected from a local slaughterhouse and placed in ice-cold saline. The aorta fragments were fixed separately with GA, TA, and a combination of GA and TA as described below. Following fixation, the samples were treated with high concentrations of elastase to test resistance to enzymatic degradation.

Porcine aortic conduits were fixed for 7 days at room temperature in either:

0.6% GA in 50 mM Hepes buffered saline at pH 7.4 (GA);

0.3% TA in 50 mM Hepes buffered saline at pH 7.4 (TA); or

A mixture of 0.6% GA and 0.3% TA in 50 mM Hepes buffered saline at pH 7.4 (GA & TA).

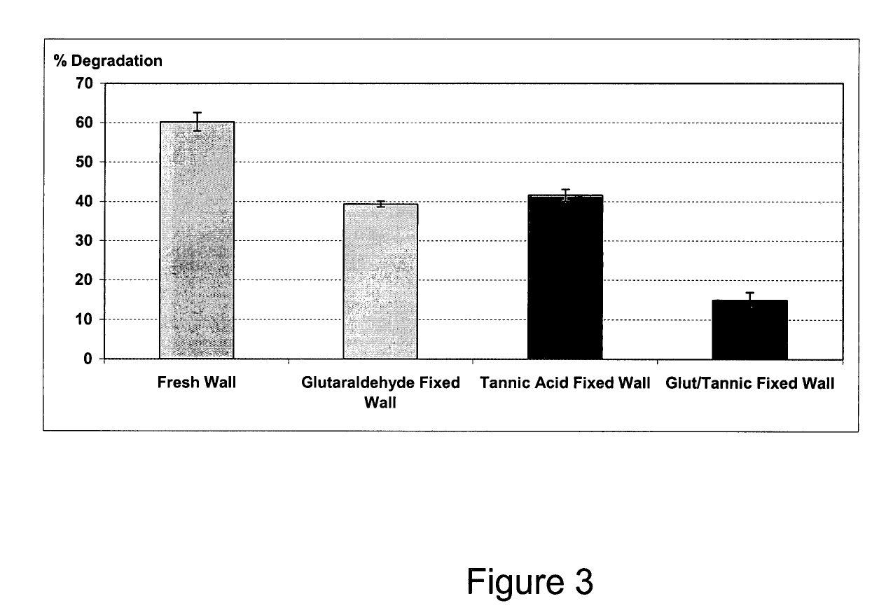

After fixation, tissues were washed in normal saline followed by ddH₂O and fragments of 4×4 mm were dissected and lyophilized. Tissue fragments from each group were weighed and incubated for 2 days (about 48 hours) at 37° C. with 8.5 Units of pancreatic elastase in elastase buffer (50 mM Tris, 1 mM CaCl₂, 0.02% NaN₃, pH 7.8). As positive controls, fresh, untreated tissues exposed to elastase solution were used. Tissue fragments were thoroughly rinsed in ddH₂O, lyophilized and weighed. Mass loss due to enzyme digestion was calculated from the difference between tissue weight before and after incubation in elastase. Lower values of Mass Loss, set forth in Table 2 and FIG. 3, are indicative of better tissue preservation and improved putative protection of elastin from enzymatic degradation.

TABLE 2
Effect of TA and GA on degradation of aortic wall

| Treatment | % Mass Loss | SEM (n = 6) |
|---|---|---|
| Fresh | 60.19 | 2.32 |
| GA | 39.33 | 0.75 |
| TA | 41.56 | 1.52 |
| GA + TA | 14.91 | 1.96 |

The results indicate that the fresh elastin-rich aortic wall tissue is susceptible to elastase (60% mass loss in 2 days) GA fixation alone, as well as TA fixation alone increased the resistance to elastase of aorta by about 20% indicating the ability of both agents to act as cross-linkers, while a mixture of GA and TA reduced mass loss more than 4-fold (from 60% to 14%). The data indicate that TA is effective in preventing elastin degeneration and that GA and TA may have additive effects in protecting biological tissues from degeneration.

### EXAMPLE 3

Collagen rich tissue was used as a model to test the possible interference of TA with GA-mediated fixation.

Samples of bovine pericardium (tissue including about 85% collagen and 5-10% elastin) was fixed at room temperature in one of either:

1% TA in phosphate buffered saline at pH 7.4 for 24 and 44 hours (TA);

0.5% GA in phosphate buffered saline at pH 7.4 for 24 and 44 hours (GA);

0.5% GA in phosphate buffered saline at pH 7.4 for 60 minutes followed by 1% TA in phosphate buffered saline at pH 7.4 for 24 and 44 hours (GA/TA); or

1% TA in phosphate buffered saline at pH 7.4 for 60 minutes followed by 0.5% GA in phosphate buffered saline at pH 7.4 for 24 and 44 hours (TA/GA).

After fixation, tissues were washed in normal saline and the extent of cross-linking was evaluated by analysis of the thermal denaturation temperature (T_{d}). T_{d} reflects that the temperature at which native collagen molecules unravel (around 65° C.) is increased in chemically cross-linked tissues.

TABLE 3
Effect of TA and GA on degradation of pure elastin

| | T_{d} after 24 hours | T_{d} after 44 hours |
|---|---|---|
| Treatment | (in ° C. +/− SEM, n = 6) | (in ° C. +/− SEM, n = 6) |
| TA | 68.4 +/− 1.4 | 72.6 +/− 0.9 |
| GA | 84.5 +/− 0.7 | 85.1 +/− 1.1 |
| GA/TA | 89.5 +/− 0.9 | 90.4 +/− 1.3 |
| TA/GAt | 84.9 +/− 0.8 | 85.2 +/− 0.9 |

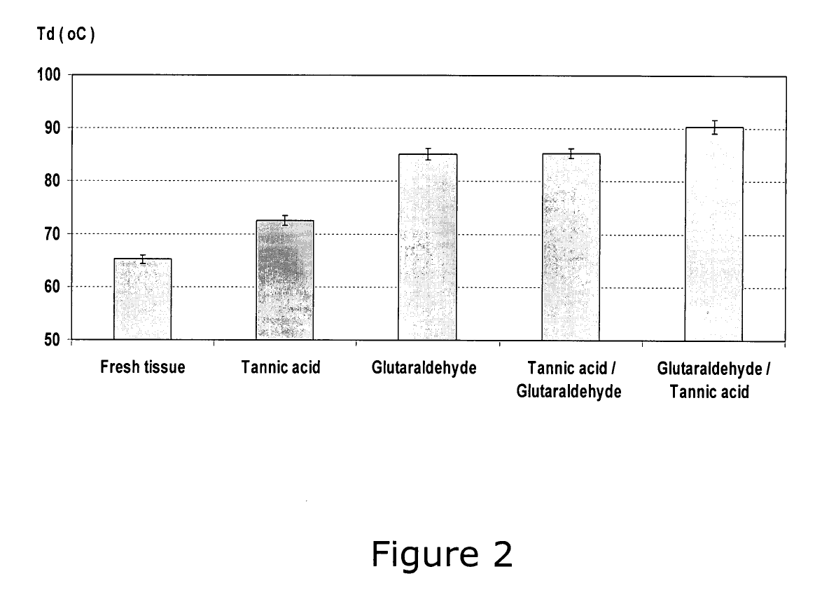

The results are shown in Table 3 and in FIG. 2. The results indicate that TA alone is only partially efficient in cross-linking collagen, while GA alone is very efficient and reaches a plateau after 24 hours. When TA follows GA, higher T_{d} values were obtained as compared to either treatment alone, indicating that TA may cross-link sites where GA has no access. When pericardium was fixed with TA first and then with GA, no apparent change was seen in Td values (as compared to GA alone), indicating that TA does not interfere with GA fixation significantly.

### EXAMPLE 4

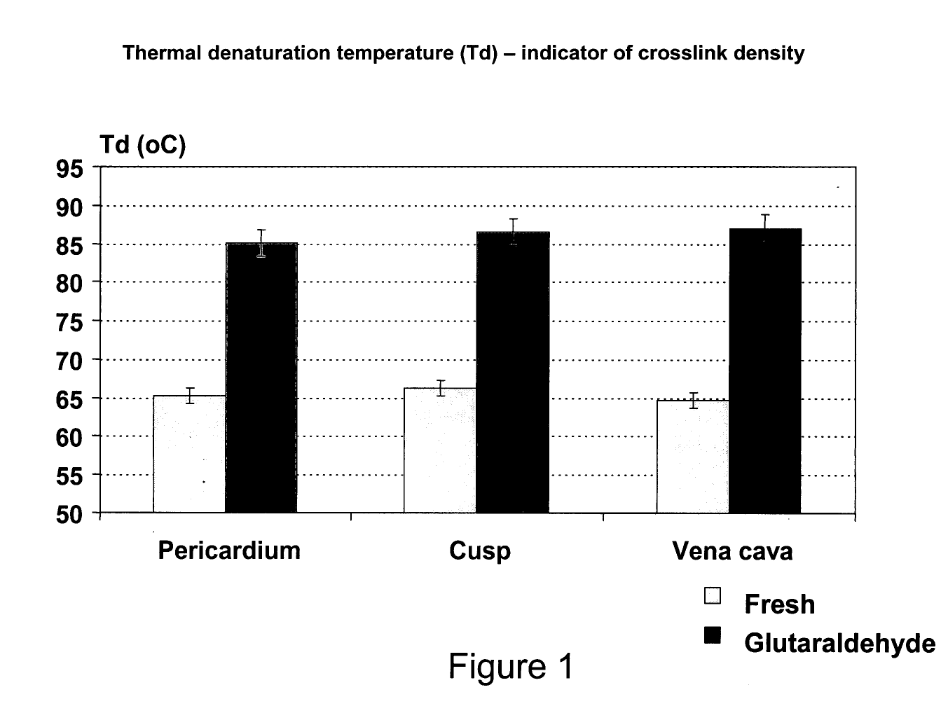

T_{d} indicates the amount of energy absorbed by a sample. In the case of connective tissues, T_{d} represents the temperature at which native collagen molecules unravel. This process leads to protein denaturation and is recorded as a peak maximum (FIG. 1). Fresh, native pericardium tissues exhibit a T_{d} of around 65° C., while chemically cross-linked tissues require a larger amount of heat to denature, and therefore their T_{d} increases proportionally to the number of cross-links.

Tissues (native, GA fixed, TA fixed, and GA/TA combination fixed, as described above) were rinsed in saline and 2 mm² samples were cut and hermetically sealed in Differential Scanning Calorimetry (DSC) aluminum pans. Samples were heated at a rate of 10° C./min, from 25° C. to 110° C. and the temperature of thermal denaturation (T_{d}) for each sample was recorded on a Perkin Elmer DSC 7 machine.

Fresh pericardium exhibited a T_{d} of around 65° C., while chemical cross-linking with GA increased T_{d} values to 87° C. (FIG. 1) indicative of a high degree of cross-linking. As best seen in reference to FIG. 2, a fixation protocol for pericardium which involves glutaraldehyde followed by tannic acid, results in a higher cross-link density. This correlates with the data seen in FIG. 3 showing increased resistance to aortic wall degradation by elastase for glutaraldehyde/tannic acid fixed wall material. It is important to note that the tannic acid fixation does not interfere diminish the beneficial effects of fixation with glutaraldehyde. In addition, the combination of glutaraldehyde fixation followed by tannic acid appears to offer improvements as opposed to treating first with tannic acid followed by glutaraldehyde. As a result, the above data suggest that tissue previously fixed with glutaraldehyde may be improved by a subsequent treatment of tannic acid. The subsequent tannic acid fixation will increase cross-link density by fixing elastin molecules which are largely unaffected by glutaraldehyde fixation.

### EXAMPLE 5

Porcine aorta fragments were collected from a local slaughterhouse and placed in ice-cold saline. The aorta fragments were fixed with GA and separately with a combination of GA and TA as described below. Following fixation, samples were implanted subdermally in juvenile rats to test for calcification potential.

Porcine aortic conduits were fixed for 7 days at room temperature in either:

0.6% GA in 50 mM Hepes buffered saline at pH 7.4 (GA); or

a mixture of 0.6% GA and 0.3% TA in 50 mM Hepes buffered saline at pH 7.4 (GA & TA).

After fixation, tissues were washed in normal saline and fragments of 4×4 mm were tested for calcification by subdermal implantation in juvenile rats (the process used is described in detail by Bailey M, Xiao H, Ogle M, Vyavahare N., ‘Aluminum chloride pretreatment of elastin inhibits elastolysis by matrix metalloproteinases and leads to inhibition of elastin-oriented calcification,’ The American Journal of Pathology, 2001;159(6):1981-6, which is herein incorporated by reference). This is a well-established experimental model in which glutaraldehyde-fixed tissue samples undergo pathologic calcification which shares many similarities to clinical specimens (bioprostheses explanted from humans due to degeneration and calcification). Moreover, calcification in the subdermal model is highly accelerated, reaching in only 3-4 weeks calcification levels observed in humans after more than 10 years post-implantation. Briefly, this procedure involves making a small incision on the back of 2-3 week-old Sprague-Dawley rats (weighing ˜50 g), creation of a subdermal pouch using blunt dissection, placement of a tissue fragment in the subdermal pouch and closure of the incision with surgical staples.

At 7 and 21 days after implantation, rats were humanely euthanized, tissue samples explanted from their subdermal pouches, dried and analyzed for calcium content using atomic absorbtion spectrophotometry (as outlined in publication above). Calcification levels obtained are expressed in Table 4, below as micrograms of calcium per mg dry explanted tissue.

TABLE 4
Effect of TA and GA on calcification of aorta in an animal model

| | Calcium content after | Calcium content after |
|---|---|---|
| | 7 days (μg Ca/mg | 21 days (μg Ca/mg |
| Treatment | dry +/− SEM, n = 10) | dry +/− SEM, n = 10) |
| GA | 15.31 +/− 1.54 | 42.18 +/− 2.21 |
| GA/TA | 5.86 +/− 0.52 | 14.68 +/− 1.05 |

The results indicate that GA-fixed aorta calcifies heavily in this model while aorta treated with a mixture of GA and TA exhibits a statistically significant (p<0.01) reduction in calcification at both time points (more than 60% reduction). The data suggest that TA is effective in inhibiting calcification of aortic segments possibly by reducing elastin degeneration.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention, which is set forth in the following claims. In addition, it should be understood that aspects of the various embodiments may be interchanged, both in whole or in part. Therefore, the spirit and scope of the appended claims should not be limited to the description of the preferred versions contained therein.

That which is claimed is:

1. A process of fixing a tissue comprising:
providing a tissue comprising elastin;
fixing said tissue with a solution comprising a phenolic tannin; and
washing said tissue, thereby providing a fixed tissue having an elastin component substantially resistant to biodegradation.

2. The process of claim 1, wherein the tissue further comprises collagen, the process further comprising fixing the tissue with a solution comprising glutaraldehyde.

3. The process of claim 2, wherein the tissue is fixed with the solution comprising a phenolic tannin subsequent to the fixing of the tissue with the solution comprising glutaraldehyde.

4. The process of claim 1, wherein the tissue is xenograft tissue.

5. The process of claim 1, wherein the tissue is selected from the group consisting of pericardium, aortic arch, heart valve, and vena cava tissue.

6. The process of claim 1, in which the phenolic tannin is tannic acid.

7. The process of claim 6, in which the solution comprising tannic acid comprises tannic acid in a concentration between about 0.0001 g/100 ml solution and about 10 g/100 ml solution.

8. The process of claim 7, in which the solution comprising tannic acid comprises a buffer, the solution being at a pH of less than about 6.

9. The process of claim 1, wherein the tissue comprises at least about 10% elastin by weight.

10. The process of claim 1, wherein the tissue further comprises glycosaminoglycan polysaccharides, the process further providing a fixed tissue wherein the glycosaminoglycan polysaccharides are substantially resistant to biodegradation.

11. A process of forming a bioprosthesis comprising:
exposing a connective tissue to a solution comprising an effective amount of a phenolic tannin, thereby chemically fixing an elastin component of the tissue; and
incorporating the fixed tissue into a bioprosthesis.

12. The process of claim 11, further comprising exposing the connective tissue to an effective amount of glutaraldehyde.

13. The process of claim 11, wherein the step of incorporating the fixed tissue into a bioprosthesis comprises attaching the fixed tissue to a support structure.

14. The process of claim 13, wherein the support structure comprises a stent.

15. The process of claim 11, wherein the bioprosthesis is a bioprosthetic heart valve.

16. The process of claim 11, wherein the connective tissue is an anisotropic material exhibiting increased elasticity in a direction, the process further comprising orienting the anisotropic material within the bioprosthesis with the direction of increased elasticity in a specific orientation such that the tissue mimics the elastic characteristics of the natural tissue which it is replacing.

17. The process of claim 11, wherein the phenolic tannin is tannic acid.

18. The process of claim 17, in which the solution comprising tannic acid comprises tannic acid in a concentration between about 0.0001 g/100 ml solution and about 10 g/100 ml solution.

19. The process of claim 17, in which the solution comprising tannic acid comprises tannic acid in a concentration between about 0.3 g/100 ml solution and about 1.0 g/100 ml solution.

20. A fixed tissue comprising cross-linked elastin, wherein the elastin is cross-linked with a phenolic tannin cross-linking agent.

21. The fixed tissue of claim 21, further comprising cross-linked collagen, wherein the collagen is cross-linked with a glutaraldehyde cross-linking agent.

22. The fixed tissue of claim 21, wherein the tissue exhibits at least about 60% less calcification over time as compared to a similar tissue fixed with only a glutaraldehyde fixative.

23. The fixed tissue of claim 20, wherein the fixed tissue comprises at least about 10% elastin by weight.

24. The fixed tissue of claim 20, wherein the phenolic tannin cross-linking agent is tannic acid.

25. The fixed tissue of claim 20, wherein the fixed tissue has a temperature of thermal denaturation greater than about 70° C.

26. The fixed tissue of claim 20, wherein the fixed tissue has a temperature of thermal denaturation greater than about 80° C.

27. The fixed tissue of claim 20, wherein the fixed tissue exhibits less than about 20% degradation following exposure to elastase for a period of about 48 hours.

28. The fixed tissue of claim 20, wherein the tissue is selected from the group consisting of bovine and porcine tissue.

29. The fixed tissue of claim 20, wherein the tissue is selected from the group consisting of pericardium, aortic wall, heart valve, and vena cava tissue.

30. A bioprosthesis comprising:
a fixed tissue comprising elastin cross-linked with a tannic acid cross-linking agent; and
a support material attached to the fixed tissue.

31. The bioprosthesis of claim 30, in which the tissue has an elastin content of greater than about 10% by weight of the tissue.

32. The bioprosthesis of claim 30, in which the tissue further comprises collagen cross-linked with a glutaraldehyde cross-linking agent.

33. The bioprosthesis of claim 30, wherein the tissue is an anisotropic tissue.

34. The bioprosthesis of claim 33, wherein the anisotropic tissue exhibits greater stiffness in a first direction and greater elasticity in a second direction.

35. The bioprosthesis of claim 30, wherein the tissue is selected from the group consisting of pericardium, aortic wall, heart valve and vena cava tissue.

36. The bioprosthesis of claim 30, wherein the tissue is porcine vena cava tissue.

37. The bioprosthesis of claim 30, wherein the support material comprises a stent.

38. The bioprosthesis of claim 30, wherein the support material comprises a suture ring.

39. The bioprosthesis of claim 30, wherein the bioprosthesis is a bioprosthetic heart valve.

40. The bioprosthesis of claim 30, wherein the bioprosthesis exhibits at least about 60% less calcification over time as compared to a similar bioprosthesis in which the tissue is fixed with only glutaraldehyde.

41. A process for replacing a damaged cardiac valve comprising:
surgical removal of a damaged cardiac valve from the heart of a patient;
implantation of a bioprosthetic heart valve in the cardiac valve annulus, wherein the bioprosthetic heart valve comprises a fixed tissue comprising elastin cross-linked with a tannic acid cross-linking agent; and
attachment of the bioprosthetic heart valve to the tissue of the cardiac valve annulus.

42. The process of claim 41, wherein the tissue has an elastin content of at least about 10% by weight of the tissue.

43. The process of claim 41, wherein the tissue further comprises collagen cross-linked with a glutaraldehyde cross-linking agent.

44. The process of claim 41, wherein the bioprosthetic heart valve is a tricuspid heart valve.

45. The process of claim 41, wherein the bioprosthetic heart valve is a bicuspid heart valve.

46. The process of claim 41, wherein the tissue is selected from the group consisting of pericardium, aortic wall, heart valve, and vena cava tissue.
